(19) United States
(12) Patent Application Publication
(10) Pub. No.: US 2009/0306663 A1
(43) Pub. Date: Dec. 10, 2009

(54) Bone Fixation Device

(76) Inventor: Gregory James Roger, New South wales (AU)

Correspondence Address: SCHWEGMAN, LUNDBERG & WOESSNER, P.A., P.O. BOX 2938, MINNEAPOLIS, MN, 55402, US

(21) Appl. No.: 12/226,580

(22) PCT Filed: Apr. 13, 2007
(86) PCT No.: PCT/AU2007/000491

§ 371 Date: Feb. 20, 2009

(30) Foreign Application Priority Data
Apr. 27, 2006 (AU) 2006902182

(51) Int. Cl. A61B 17/56

(52) U.S. Cl. 606/63; 606/62

(57) ABSTRACT

A bone fixation device and a method of securing the same in a fractured bone. The device has a rod and at least one bone engaging member The rod and at least one bone engaging member bridge a region of fracture with the end of the at least one bone engaging member engaging with the bone of a patient to secure the device across the fracture.

### FIELD OF THE INVENTION

The present invention relates to a device and method for stabilising and aligning fractured fragments of a bone.

### BACKGROUND OF THE INVENTION

Fractures of long bones, such as the tibia, femur and humerus, are a common occurrence. Due to the forces involved in the fracture and the forces normally carried by the long bones, these fractures are often unstable and the fracture pattern complex.

There are several methods of treating such fractures including external bracing and plaster casts. Some fractures require open reduction and internal fixation with plates applied to the exterior of the bone and secured with screws across the bone. In other cases, external fixateur is used with rods passed through the long bone fragments and held in position by a frame external to the skin of the patient. A still further means of stabilising and aligning a fracture is the use of intra-medullary (IM) nails.

IM nails are typically introduced into the medullary cavity of a proximal fragment of bone, passed down through the medullary canal and across the fracture to bridge the fracture and control the proximal and distal fragments of bone in terms of rotation and bending.

IM nails are typically secured at their distal ends by external screws. The external screws are inserted through stab wounds in the skin of a patient adjacent to the distal end of the IM nail. The screws are advanced through holes in the IM nail to distally fix the nail. In order to align the screws with the IM nail holes in the distal bone fragment, an Image Intensifier (II) X-ray is used. The use of such a machine is complex, requiring specialised staff, not to mention time consuming and expensive.

The present invention aims to address the problems associated with securing an IM nail in a bone and particularly the problems associated with securing an IM nail distal to the site of a fracture.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY OF THE INVENTION

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In a first aspect, the present invention consists in a bone fixation device, the bone fixation device including a rod member extending along a longitudinal axis from a proximal end to a distal end; and at least one bone engaging member that is longitudinally movable relative to the rod member between a first delivery position and a second bone engaging position, the rod member having one or more guide members to guide the at least one bone engaging member to its second bone engaging position.

In a second aspect, the present invention provides a method of securing a bone fixation device in a fractured bone of a patient; the bone fixation device including a rod member that extends along a longitudinal axis from a proximal end to a distal end and at least one bone engaging member, said at least one bone engaging member extending from a first end to a second end, the method including the steps;

(i) introducing the rod member and the at least one bone engaging member into the medullary space of a fractured bone;

(ii) advancing the rod member and the at least one bone engaging member through the medullary space and across the site of fracture such that the proximal end of the rod member and the first end of the at least one bone engaging member are positioned proximal to the fracture site and the distal end of the rod member and the second end of the at least one bone engaging member are positioned distal to the fracture site; and

(iii) further advancing the at least one bone engaging member and causing at least the second end of the at least one bone engaging member to extend into a portion of cortical bone distal to the site of fracture.

The rod member may be cylindrical and substantially straight along a majority of its length. However, it is also envisaged that the rod member may vary in diameter along its length whilst still retaining a generally straight configuration along its longitudinal axis. In a preferred embodiment, the rod member comprises a substantially straight main shaft and a distal end portion wherein the distal end portion is greater in diameter than the greatest diameter of the main shaft.

The at least one bone engaging member may comprise at least one pin member. Preferably, the at least one bone engaging member comprises two pin members. An embodiment wherein the device comprises three or more pin members is also envisaged.

The pin members may extend from a first end to a second end and are longitudinally moveable relative to the rod member. The pin members may be substantially straight along their length when they are in their first delivery position. The pin members may have a constant diameter along their length or they may vary in diameter along their length.

In their bone engaging position, the pin members typically have an angled configuration. In this embodiment, a portion of a pin member at, or substantially adjacent to, the second end may be angled relative to the remainder of the pin member. The second end of each pin member may be tapered to form a point. Further, a portion of each pin member adjacent to the distal end of the second end may be screw threaded.

When they are in their delivery position, the pin members may be substantially longitudinally aligned with the rod member. The pin members may engage the rod member when they are in their delivery position or, alternatively, the pin members may be relatively spaced from the rod member in their delivery position.

Typically, the distal end portion of the rod member comprises a blunt ended bulb member. Further, the distal end portion of the rod member preferably comprises the one or more guide members. In the embodiment wherein the device includes two pin members, the distal end portion preferably comprises two guide members. The number of guide members may vary, particularly depending upon the number of pin members of the device. Reference below will be made to two guide members and two corresponding pin members.

The two guide members may include two channels in an outer surface of the distal end portion. Each channel may receive a bone engaging portion of a corresponding pin member that may be moveable along said channel. The channels typically extend outwardly from and at an angle relative to the longitudinal axis of the rod member. In this embodiment, when the device of the present invention is in situ within a bone, the channels extend away from the rod member and towards the cortical bone surrounding the device.

Alternatively, the guide members may include two tunnels through the interior of the distal end portion, said tunnels extending from an opening in a surface of the distal end portion substantially adjacent to the main shaft to an opening on a surface of the distal end portion, distal to the main shaft. The bone engaging portion of a corresponding pin member is moveable through each tunnel. The tunnels typically extend along their length at an angle relative to the longitudinal axis of the rod member. In this embodiment, when the device is in situ within a bone, the tunnels extend towards the cortical bone surrounding the device.

In a further embodiment, the one or more guide member may comprise at least a portion of the external surface of the distal end portion. As noted above, the diameter of the distal end portion is greater than the diameter of the greatest diameter of the main shaft. The distal end portion is typically a bulb member that presents an angled surface relative to the main shaft. Such an angled surface may act as a guide member for a pin member as will be discussed in further detail below.

The pin members may be delivered to a fracture site by the rod member or by an introducer collar.

In the embodiment wherein the pin members are delivered by the rod member, when in their delivery position the pin members may engage an outer surface of the rod member and extend along a length of the rod member from its proximal end to a region of the rod member just proximal to the distal end portion. The rod member may include receiving members to engage the pin members along said length. The receiving members may be channels in the external surface of the rod member. Each channel may align with a corresponding guide member of the distal end portion such that the pin members are moveable between the channels and the guide members.

Alternatively, the receiving members of the rod member may comprise tunnels through the interior of the rod member; A pin member may be moveable through a corresponding tunnel from the proximal end of the rod member to a region of the rod member just proximal to the distal end portion of the rod member. In this embodiment, each tunnel may align with a corresponding guide member of the distal end portion such that the pin members are moveable between the tunnels and the guide members.

In the above embodiments, the rod member and the pin members may be moved through a medullary space in a bone together and to a position where the rod member and the pin members bridge a site of fracture.

In the embodiment of the invention wherein the pin members are delivered by an introducer collar, the pin members are relatively spaced from the rod member in their delivery position. Whilst there may be a degree of engagement between the pin members and the rod member as the pin members are longitudinally moved relative to the rod member, it should be understood that the pin members are carried by the introducer collar through the medullary space of a bone and not by the rod.

The introducer collar may comprise a tubular main body that is slidably moveable over the rod member.

The tubular main body may include two receiving members to receive the pin members. The receiving members may comprise passages through the interior of the main body of the introducer collar. The passages may extend from an opening in an upper surface of the tubular main body to an opening in a lower surface of the tubular main body.

The introducer collar may further include an elongate handle that enables positioning of the introducer collar relative to the rod member when the device is in use. In this regard, the introducer collar may be both longitudinally moveable relative to the rod member and also rotationally moveable around the rod member. The latter feature has the advantage that the introducer collar may be rotated around the rod member such that the receiving members of the distal locking member are substantially aligned with corresponding guide members of the distal end portion of the rod member. A pin member may thus be moveable between the receiving member of the introducer collar and the corresponding guide member.

The device of the present invention may further include a locking member. The locking member may comprise two components, a distal locking member and a proximal locking member which together secure the rod and pin members within a fractured bone when the device is in use.

The distal locking member may comprise an elongate tubular main body that extends from a first end to a second end and has an inner lumen sufficiently sized to receive the rod member and the pin members. Typically, the distal locking member is shorter in length than the rod member.

An inner surface of the distal locking member may include holding members to engage the pin members. The holding members may comprise two longitudinal channels.

The distal locking member may be longitudinally moveable relative to the rod member from the proximal end of the rod member towards the distal end portion. Typically, the diameter of the lumen of the distal locking member is smaller than the diameter of the distal end portion thus preventing further movement of the distal locking member beyond the distal end portion.

The distal locking member may be moveable between a non-locked configuration and a locked configuration. In the non-locked configuration, the distal locking member may be slidably moveable relative to the rod member and the pin members. In its locked configuration, the second end of the distal locking member may be in engagement with and, preferably, in tight abutment with distal end portion of the rod member.

The distal locking member may comprise a continuous tubular main body or, alternatively, a number of inter-connectable tubular segments. The diameter of the distal locking may vary along its length.

The distal locking member is held in its locked position by the proximal locking member. The proximal locking member may also comprises an elongate tubular main body having a lumen to receive at least a portion of the rod member. The proximal locking member may have an internal screw threaded portion that is engageable with a complementary screw threaded portion at and substantially adjacent to the proximal end of the rod member. The proximal locking member may thus be screw threadedly movable along a length of the rod member from its proximal end towards its distal end. In use, the distance along the rod member that the proximal locking member may be advanced is determined by the positioning of the distal locking member. As the proximal locking member is advanced towards the distal end of the rod member, it may be brought into engagement with the first end of the distal locking member. The movement of the proximal locking member applies a force on the distal locking member such that its second end may be brought into tight abutment with the distal end portion of the rod member. The proximal locking member may be secured within a bone of a patient by its screw threaded engagement with the rod member. The device of this embodiment, may further include an additional locking nut to secure the proximal locking member.

In another embodiment, the proximal locking member may slide over the proximal end of the rod member and be secured by a locking member. The locking member may comprise a locking pin that engages a keyway in the proximal locking member. In this regard, the locking pin typically includes a flanged portion that is received within a longitudinal recess of an inner wall of the proximal locking member. The locking pin is, therefore, slidable moveable along a length of the proximal locking member and, preferably, along the entire length of the proximal locking member. The locking pin may include an extension member that is receivable in a slot on the proximal end of the rod member. The device of this embodiment may further include a locking nut that is positioned proximal relative to the locking pin and which is screw threadedly moveable through the lumen of the proximal locking member until it is brought into engagement with the locking pin. Further screw threading of the locking nut forces the locking pin into tight engagement with both the rod member and the first end of the distal locking member.

The pin members of the device may be made from a resiliently flexible material. An example of a suitable material is a shape memory material including, but not limited to Nitinol™.

The rod member may be of varying lengths and may be determined by the desired length of fixation of the bone. Typically, the pin members are longer than the rod member.

The distal locking member may be made of Titanium alloy. Alternatively, it may be made of a resorbable material such as BioGlass or a Hydroxyapatite composite. The distal locking member may also be made of a number of materials, for example it may be partly made from a Titanium alloy and partly made form a resorbable material.

The distal locking member may further include anti-infective elements, including antibiotics and it may further include a furanone coating. The distal locking member may also include bone growth factors

The device of the present invention may be used to stabilise and align the fragments of a fractured bone. Particularly, the device may be used to stabilise and align a fracture of a long bone including, but not limited to, the femur, tibia, and humerus.

The rod member of the device is insertable into and through the medullary space of a fractured bone. Typically, the medullary space will have been reamed prior to insertion of the rod member. Alternatively, the rod member may be advanced through the medullary space and a reamer advanced over it to ream a space of a required diameter.

The rod member is positionable across the site of a fracture and may be secured both distal to and proximal to the site of fracture.

The pin members of the device of the present invention may secure the rod member to the distal fragment of bone.

The pin members may be advanced through the medullary space with the rod member or, as described above, delivered to the fracture site by an introducer collar. Either way, the rod and pin members are both ultimately positioned across the fracture.

The second end of each pin member is typically aligned with a corresponding guide member of the distal end portion. Once the pin members are in position they are advanced further by drilling. Because the guide members are angled towards the surrounding cortical bone, advancing the pin members along or through the guide members causes at least the bone engaging portion of the pin members to angle towards the cortical bone and eventually into the cortical bone thus securing the rod member distal to the site of fracture. Each pin member is typically advanced into the surrounding cortical bone individually. The length of each pin member that extends into the cortical bone largely depends upon the depth of the surrounding cortical bone. Preferably, a sufficient length of each pin member extends into the cortical bone to secure the device distal to the site of fracture and prevent rotation and bending of the fragments of bone.

Once the pin members are anchored in the cortical bone, the introducer collar, if used, may be withdrawn. The distal locking member may then be introduced over the proximal end of the rod member and advanced towards the distal end of the rod member until its second end abuts with the distal end portion. The proximal locking member may then be advanced over the rod member and brought into tight abutment with the distal locking member.

The feature of the invention that the pin members anchor the device distal to the fracture site from within the medullary cavity has the advantage that external screws (inserted through stab wounds in the skin and then through apertures in the rod member) need not be used.

In a further embodiment, the pin members may extend through the cortical bone and out of the surrounding skin. The second end of each pin member may act as a chaser tip for screws. The screws may engage the pin members and be driven back in through the skin and soft tissue, through the cortical and then engage the rod member to secure said rod member within the bone. The screws may extend through the rod and engage an opposite portion of cortical bone. The pin members may then be removed, Whilst still using external screws, this embodiment still has the advantage that complex imaging is not needed to align the screws with the rod member. Further, this embodiment may be useful where the distal locking collar is made from a resorbable material.

### BRIEF DESCRIPTION OF THE INVENTION

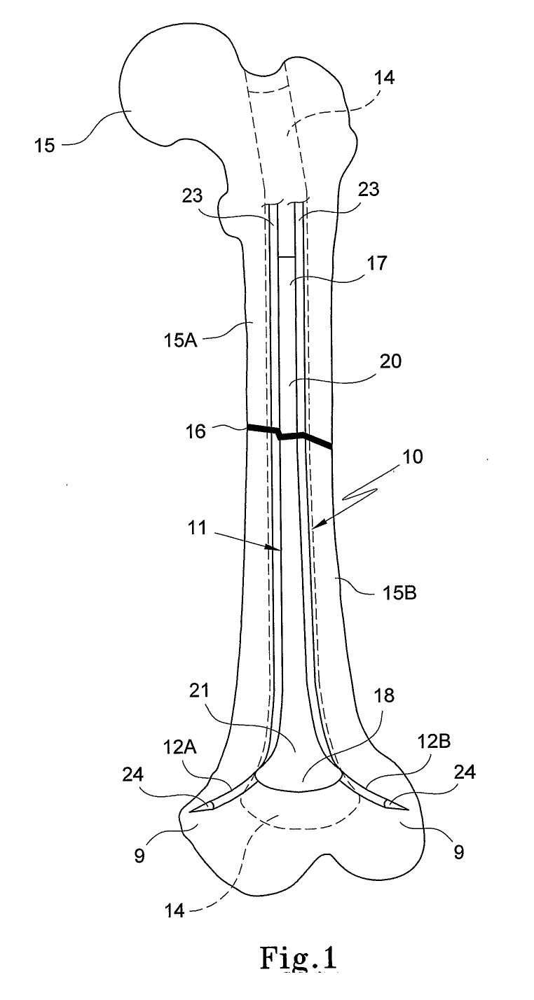

FIG. 1 is a schematic representation of the device of the present invention positioned within the medullary space of a fractured femur;

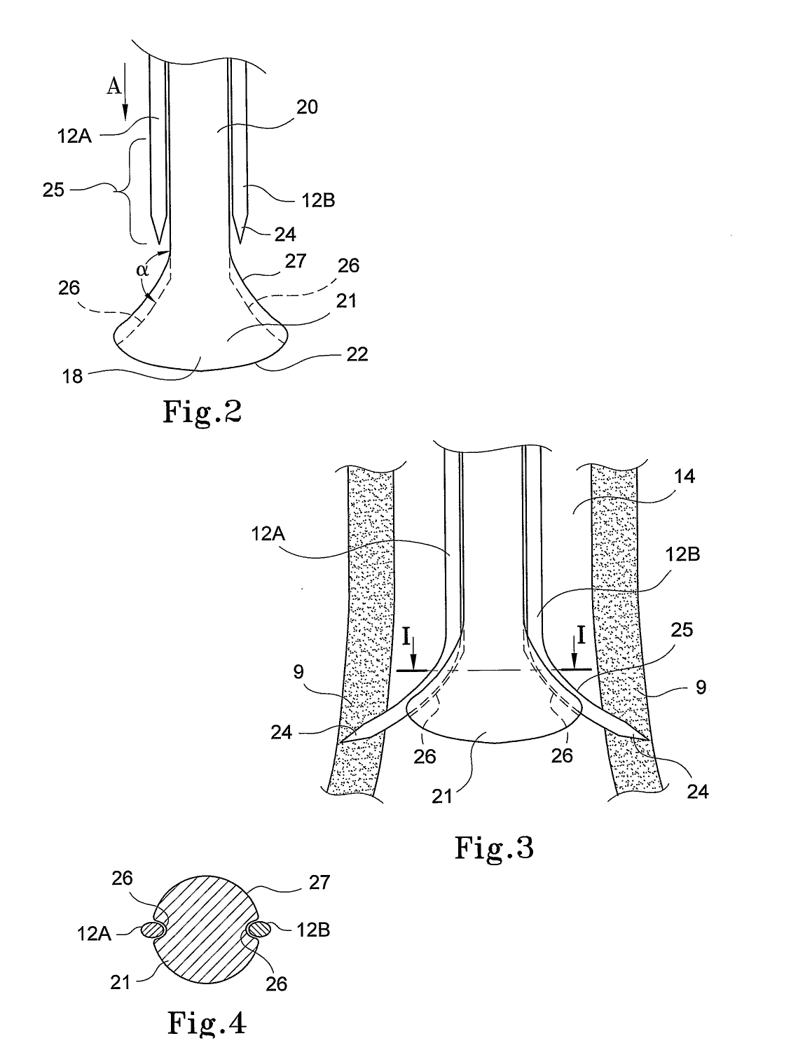

FIG. 2 is a schematic representation of an embodiment of the invention;

FIG. 3 is a further schematic view of the embodiment of the invention depicted in FIG. 2;

FIG. 4 is a cross sectional view through I-I of FIG. 3;

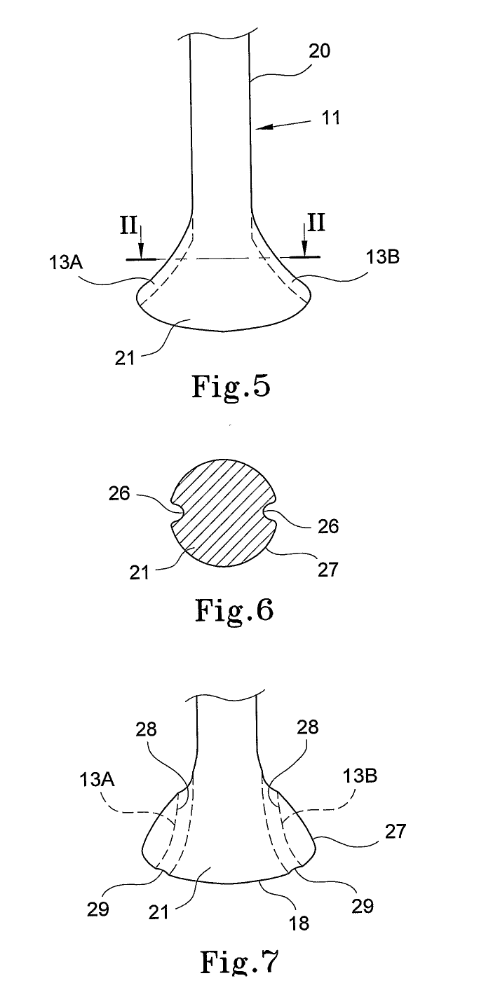

FIG. 5 is a schematic representation of a further embodiment of the invention;

FIG. 6 is a cross sectional view through II-II of FIG. 5;

FIG. 7 is a schematic view of another embodiment of the invention;

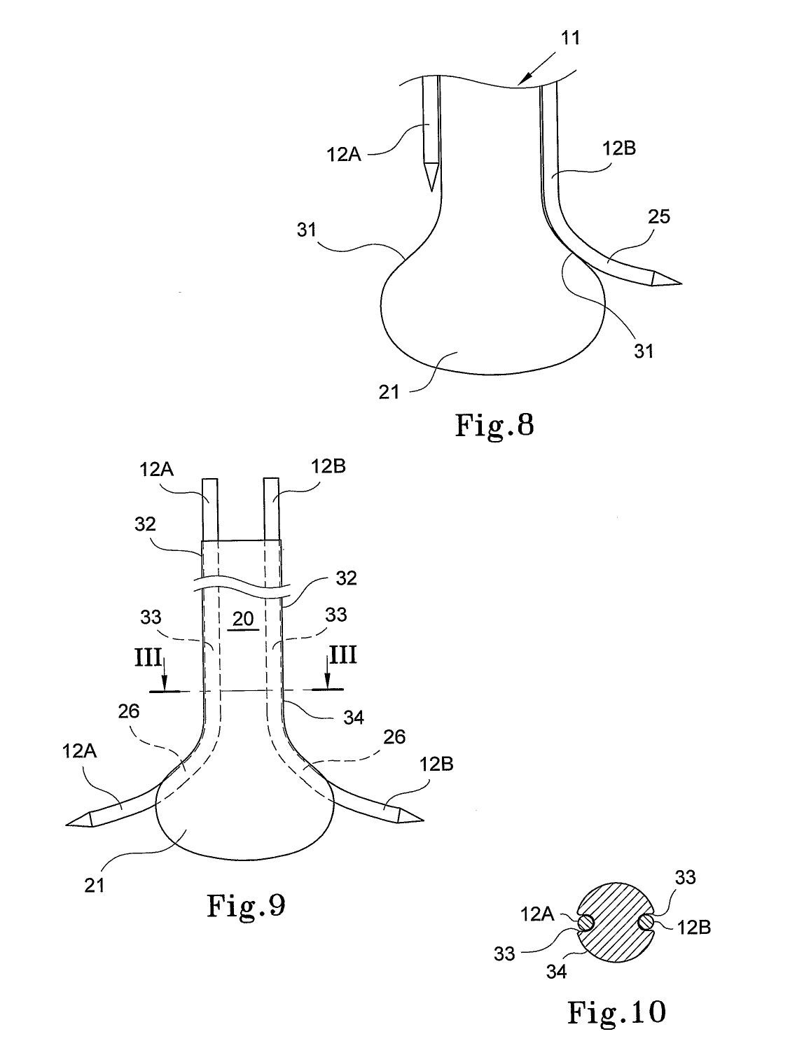

FIG. 8 represents a still further embodiment of the invention;

FIG. 9 is a schematic view of another embodiment of the invention;

FIG. 10 is a cross sectional view through III-III of FIG. 9;

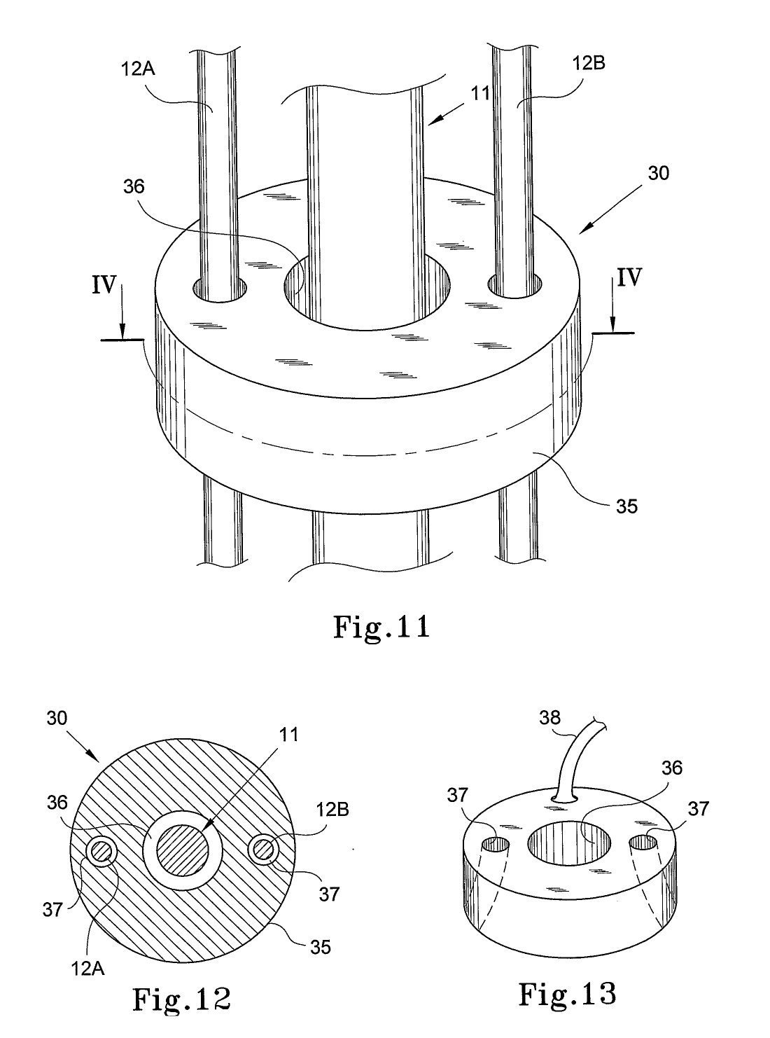

FIG. 11 is side elevational view of another embodiment of the invention;

FIG. 12 is a cross-sectional view through IV-IV of FIG. 11

FIG. 13 is a side elevational view of the introducer collar of the present invention;

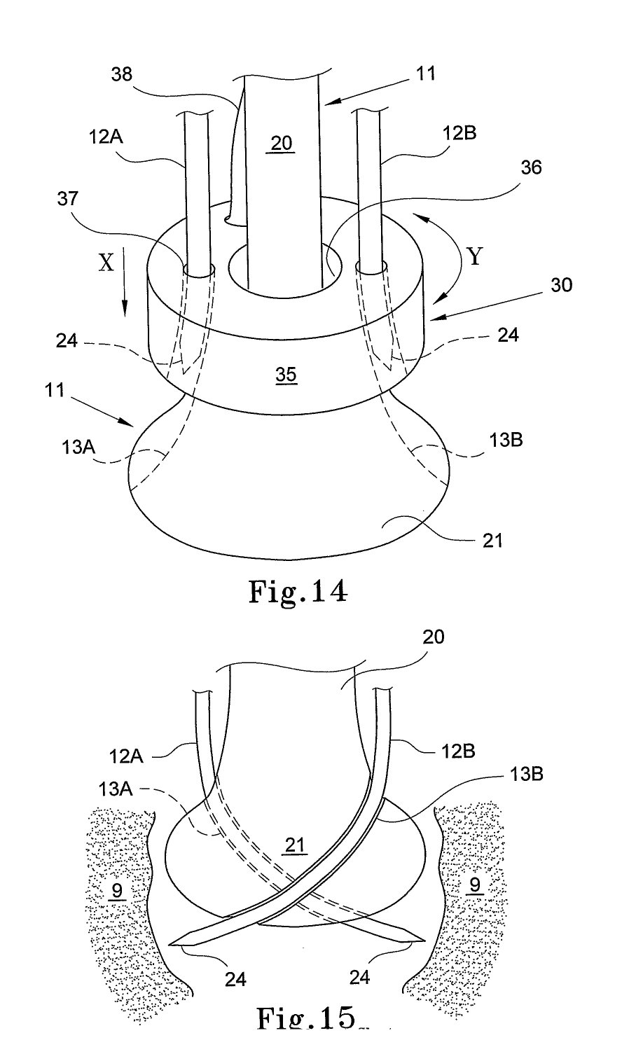

FIG. 14 is a schematic representation of a further embodiment of the invention;

FIG. 15 is a schematic view of part of the device of the present invention;

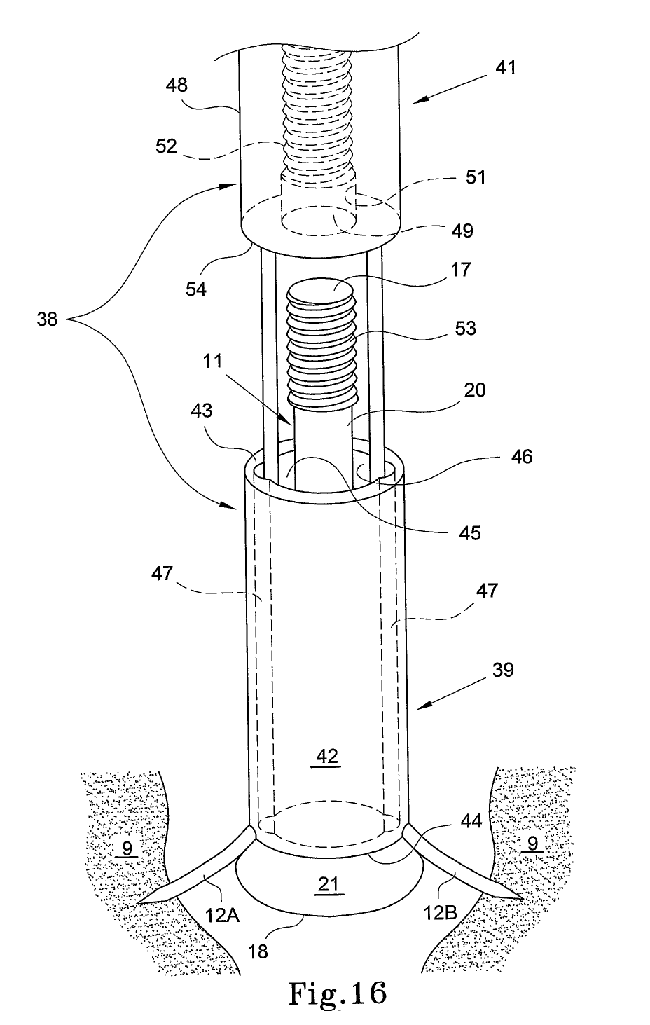

FIG. 16 is a schematic view of various components of the device;

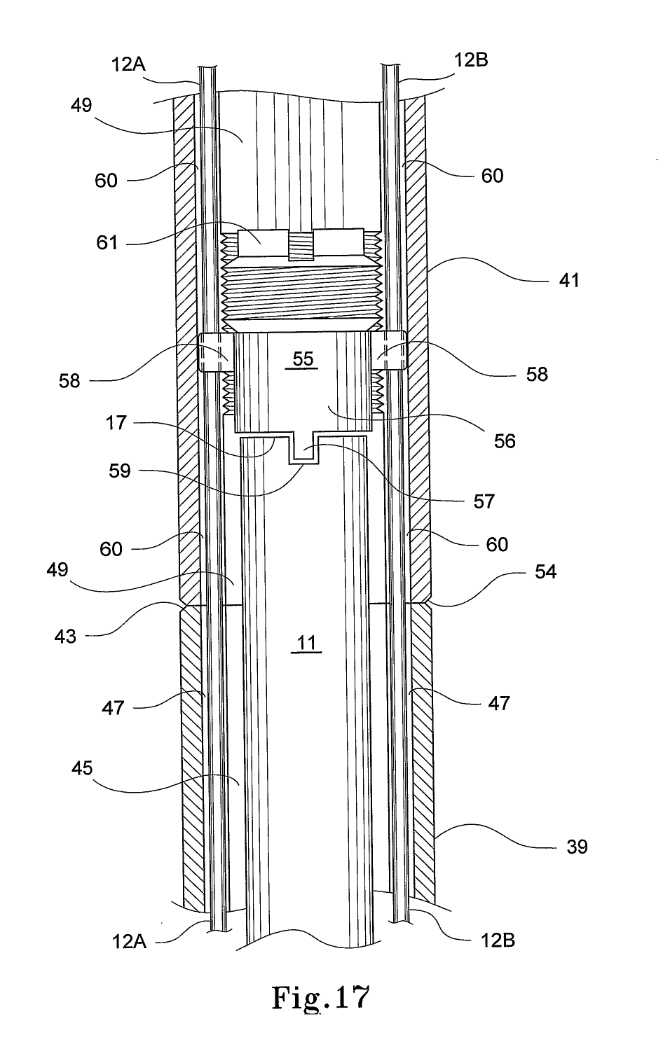

FIG. 17 is a sectional view of another embodiment of the invention;

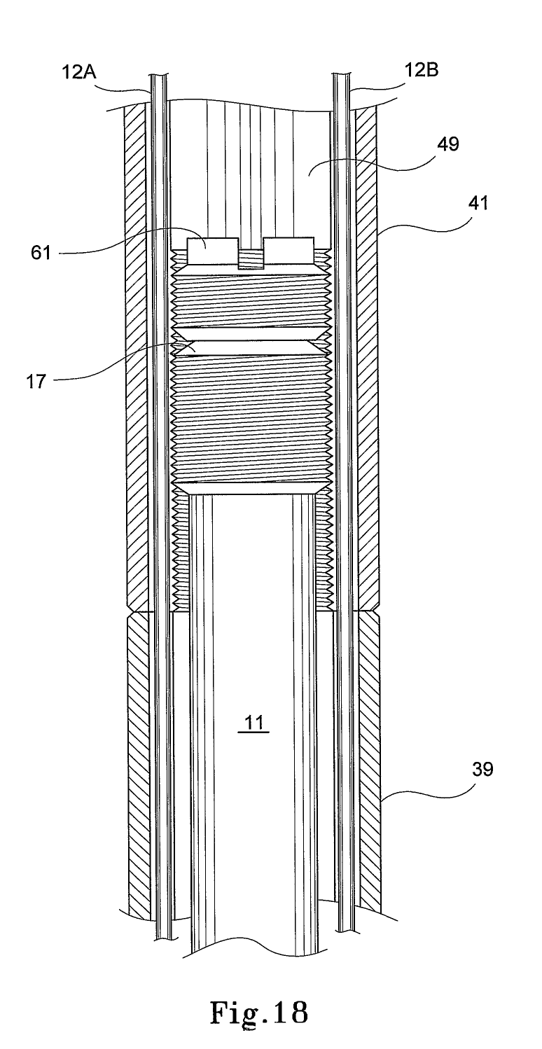

FIG. 18 is a sectional view of a further embodiment of the invention; and

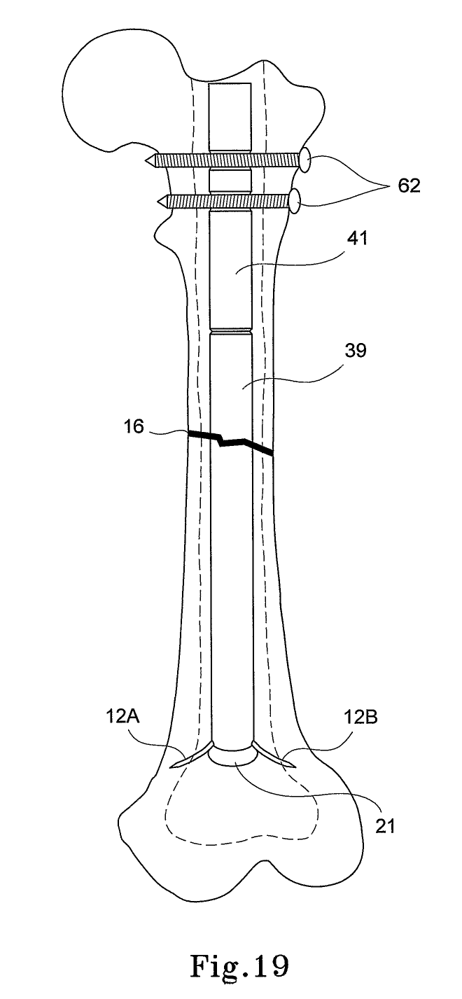

FIG. 19 is a schematic view of the device of the present invention in situ within a femur of a patient.

### DESCRIPTION OF AN EXEMPLARY EMBODIMENT OF THE INVENTION

The bone fixation device of the present invention is generally represented as 10 in the accompanying drawings.

The bone fixation device 10 comprises a rod 11 and two pins 12a and 12b. The pins 12a and 12b are movable relative to rod 11 between a first inactive position which is depicted in FIG. 2 and a second bone engaging position which can be seen in FIGS. 1, 3, 16 and 19.

The rod 11 has two guide means, 13a and 13b to guide pins 12a and 12b into their bone engaging position.

In FIG. 1, the bone fixation device 10 is shown inserted through a medullary space 14 of a femur 15. The device 10 bridges a fracture 16 to stabilise and align the proximal fragment 15a and the distal fragment 15b relative to one another. The pins 12a and 12b are inserted into the cortical bone 9 of distal fragment 15b of the fractured bone from within the medullary space 14 of the bone to secure the device distal to the fracture 16.

Rod 11 is substantially cylindrical and extends from a proximal end 17 to a distal end 18. As depicted, the rod is substantially straight along a main shaft 20 with a distal end portion 21 having a greater diameter than the main shaft 20. The distal end portion 21 comprises a blunt-ended bulb 22.

The pins 12a and 12b extend from a first end 23 to a second end 24 and are longitudinally moveable relative to rod 11. The pins 12a and 12b are shown in their straight, delivery position in FIG. 2.

In use, the pins 12a and 12b are either introduced through the medullary space with rod 11 or moved through the space separately after rod 11 is in position across the fracture. Either way, when rod 11 and pins 12a and 12b are appropriately positioned bridging the fracture, pins 12a and 12b may be longitudinally moved relative to rod 11 and in a direction toward the distal end 18 of rod 11 as shown by arrow A in FIG. 2.

The second end 24 of each pin engages part of the distal end portion 21 and is moved to its bone engaging position as depicted in FIG. 3. The advancing of pins 12a and 12b is controlled by the surgeon and, typically, each pin is advanced separately by drilling. Pins 12a and 12b are guided to the correct orientation to engage the surrounding cortical bone 9 by guide members 13a and 13b.

Guide members 13a and 13b comprise two channels 26 in an outer surface 27 of the distal end portion 21. Each channel 26 receives an end portion 25 of a corresponding pin. The channels 26 extend outwardly from and at an angle (α) relative to the longitudinal axis of rod 11.

In the embodiment depicted in FIG. 7, the guide members 13a and 13b comprise two tunnels 28 through the interior of the distal end portion 21. Each tunnel 28 opens in an aperture 29 on an outer surface 27 of distal end 18. The end portion 25 of a corresponding pin member is moveable through each tunnel 28.

In a further embodiment, depicted in FIG. 8, the guide members 13a and 13b comprise a guide area 31 on the outer surface 27 of the distal end portion 21. The pins 12a and 12b are typically made from a suitably flexible material such that as they are advanced onto the guide area 31, the end portion 25 of each pin 12a and 12b is caused to bend relative to the remainder of the length of the pin. The end portion 25 bends outwardly and away from rod 11.

The rod 11 depicted in FIG. 9 has two receiving members 32 to receive and engage pins 12a and 12b. The receiving members are channels 33 in the external surface 34 of the main shaft 20. Each channel 33 aligns with a corresponding channel 26 in the distal end portion 21.

The above description relates to embodiments of the invention wherein the pins 12a and 12b are introduced to a fracture site together with rod 11. The embodiment of the device 10 depicted in FIG. 11 comprises an introducer collar 30. The introducer collar 30 carries the pins 12a and 12b to the site of fracture.

The introducer collar 30 is a tubular main body 35 surrounding a lumen 36. The lumen 36 receives rod 11 when the device is in use.

The tubular main body 35 has two passages 37 to receive pins 12a and 12b. The passages 37 extend outwardly and away from the lumen 36 of the tubular main body 35.

The introducer collar 30 further comprises an elongate handle 38 that extends upwardly and away from the tubular main body 35.

The introducer collar 30 is longitudinally moveable relative to rod 11 and may also be rotated around rod 11 to allow alignment between the pins 12a and 12b that are carried by the introducer collar 30 and the guide members 13a and 13b of rod 11.

As depicted in FIG. 14, introducer collar 30 is moved over rod 11 towards distal end 18 as depicted by arrow X. Lumen 36 of introducer collar is smaller in diameter than the diameter of distal end portion 21 and, therefore, the introducer collar cannot move longitudinally relative to rod 11 beyond distal end portion 21. When the introducer collar 21 is in engagement with distal end portion 21, pins 12a and 12b are in a position just proximal to the guide members 13a and 13b of distal end portion 21. The surgeon may need to rotate the introducer collar 21 by way of handle 38 to fully align the pins with guide members 13a and 13b.

With pins 12a and 12b suitably aligned with guide members 13a and 13b, the surgeon may advance each pin along or through the guide members 13a and 13b. The guide members cause the end portion 25 of pins 12a and 13a to travel in a direction away from rod 11 and towards the surrounding cortical bone 9. Further drilling of each pin 12a and 12b, causes the second end 24 of each pin to engage the cortical bone and eventually enter the cortical bone until a length of the pin adjacent second end 24 is implanted within the cortical bone. The actual length of pin implanted depends upon the thickness of the surrounding cortical bone.

With both pins implanted in the surrounding cortical bone, the introducer collar is withdrawn by the surgeon.

In another embodiment, the guide members 13a and 13b of distal end portion 21 cause the end portion 25 of pins 12a and 12b to cross over the distal end portion 21 as depicted in FIG. 15. This embodiment has the advantage that the second end 25 of each pin is positioned at a more transverse angle relative to the surrounding cortical bone as it enters the bone thereby optimally securing the pin in the distal bone fragment.

In a further embodiment, the device 10 includes a locking member 38. Locking member 38 comprises a distal locking member 39 and a proximal locking member 41.

The distal locking member 39 comprises an elongate tubular main body 42 that extends from a first end 43 to a second end 44. The inner lumen 45 is sufficiently sized to receive the rod 11 and the pins 12a and 12b. The distal locking member 39 is shorter in length than rod 11 which is relevant to the positioning of the components of the device as will be discussed in further detail below.

An inner surface 46 of the distal locking member 39 includes two locking channels 47 that extend from the first end 43 to the second end 44.

In use, the distal locking member 39 is slidably moved over the rod 11 from its proximal end 17 towards the distal end 18. The distal locking member 39 is moved to a position wherein the second end 44 engages the distal end portion 21 of rod 11. The diameter of the lumen 45 of distal locking member 39 is smaller than the diameter of the distal end portion 21 and therefore, the distal locking member 39 is prevented from advancing beyond the distal end portion 21.

When the device is in situ, the distal locking member 39 surrounds a substantial length of the main shaft 20 of rod 11 and pins 12a and 12b as shown in FIG. 16.

The proximal locking member 41 comprises an elongate tubular main body 48 that extends from a first end to a second end 54. Lumen 49 of proximal locking member 41 receives a portion of the rod member 11 adjacent proximal end 17. An inner wall 51 of a portion of the proximal locking member at and adjacent to second end 54 is screw threaded. This screw threaded portion 52 of the proximal locking member 41 engages a complementary screw threaded portion 53 at and adjacent to proximal end 17 of rod 11.

When the distal locking member 39 is in position with its second end 44 engaging the distal end portion 21, a length of the main shaft 20 of rod 11 extends beyond the first end 42 of the distal locking member (because the distal locking member is shorter than the rod). The proximal locking member 41 is then advanced over the exposed proximal end 18 of rod 11 and screwed onto the complementary screw threaded portion 53 on rod 11. As the proximal locking member 41 is screw threadedly advanced, the second end 47 of proximal locking member 41 is forced down onto the first end 43 of the distal locking member 39 until the second end 44 of the distal locking member tightly abuts distal end portion 21. As depicted in FIG. 18, a lock nut 61 can be screwed through lumen 49 and brought into tight abutment with the proximal end 17 of rod 11 to further secure the components of the device 10 within the bone of a patient.

Alternatively, as depicted in FIG. 17, the proximal locking member 41 slides over the proximal end 18 and a portion adjacent thereto of rod 11 rather than screw over as described above. In this embodiment, the device includes a locking pin 55. The locking pin 55 comprises a main body 56 that has a pin member 57 depending therefrom.

The proximal locking member 41 slides over the proximal end of rod 11 until second end 54 of the proximal locking member 41 engages first end 43 of distal locking member 39. The locking pin 55 is then advanced through lumen 49 of proximal locking member 41. In this regard, the locking pin 55 has a flange 58 that engages a complementary receiving slot 59 on inner wall 51 of proximal locking member 41. The locking pin is advanced until the depending pin member 57 is received within a slot 59 in the proximal end 17 of rod 11

A lock nut 61 is screw threaded along inner wall 51 of the proximal locking member, proximal to the locking pin 55. The lock nut is brought into tight abutment with the locking pin 55 to thereby secure the proximal locking member to the rod 11 and the distal locking member 39.

The two components of the locking member 38 together aid in securing the rod 11 and pins 12a and 12b in the medullary space 14. The device is secured in the distal fragment of bone by pins 12a and 12b which are drilled into the surrounding cortical bone from within the medullary space. The feature that the pins extend from within the space 14 to the cortical bone negates the need to secure the rod by using external nails which require complex imaging to ensure that they align with corresponding holes in the rod.

The proximal locking member 41 may be secured by transverses locking screws 62 that are inserted through transverse holes 63 in the proximal locking member under direct vision and using a standard drill guide system.

If removal of device 10 is required then only a proximal approach is necessary. The proximal locking member 41 is exposed and the transverse screws 62 are removed. If used, the lock nut 61 and locking pin 55 are also removed. The proximal locking member 41 is then screwed off rod 11 and the distal locking collar removed (unless it is made from a resorbable material and has thus been resorbed). The pins 12a and 12b are then located and backed out under drill power. Finally, rod 11 is removed.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A bone fixation device including a rod member extending along a longitudinal axis from a proximal end to a distal end and at least one bone engaging member longitudinally movable relative to the rod member between a first delivery position and a second bone engaging position, the rod member having one or more guide members to guide the at least one bone engaging member to its second bone engaging position.

2. The bone fixation device of claim 1 wherein the rod member is cylindrical.

3. The bone fixation device of claim 1 wherein the rod member is substantially straight along a majority of its length.

4. The bone fixation device of claim 1, wherein the rod member comprises a main shaft and a distal end portion wherein the distal end portion is greater in diameter than the diameter of the main shaft.

5. The bone fixation device of claim 1, wherein said at least one bone engaging member comprises one or more pin members.

6. The bone fixation device of claim 1, wherein the at least one bone engaging member extends from a first end to a second end.

7. The bone fixation device of claim 1, wherein the at least one bone engaging member is substantially straight along its length when in the first delivery position.

8. The bone fixation device of claim 1, wherein the at least one bone engaging member has a constant diameter along its length.

9. The bone fixation device of claim 1, wherein the at least one bone engaging member has a variable diameter.

10. The bone fixation device of claim 1, wherein the at least one bone engaging member comprises an angled portion at or adjacent to the second end.

11. The bone fixation device of claim 1, wherein the second end of said at least one bone engaging member is tapered.

12. The bone fixation device of claim 1, wherein the at least one bone engaging member comprises a screw threaded portion.

13. The bone fixation device of claim 12 wherein said screw threaded portion is adjacent to the second end of the at least one bone engaging member.

14. The bone fixation device of claim 4, wherein the distal end portion of the rod member comprises a blunt ended bulb member.

15. The bone fixation device of claim 4, wherein the distal end portion of the rod member comprises the one or more guide members.

16. The bone fixation device of claim 15 wherein the one or more guide members comprise channels in an outer surface of the distal end portion.

17. The bone fixation device of claims 15 wherein the one or more guide members comprise tunnels through the distal end portion.

18. The bone fixation device of claim 17 wherein said tunnels extend from an opening in a surface of the distal end portion substantially adjacent to the main shaft to an opening in a surface of the distal end portion, distal to the main shaft.

19. The bone fixation device of claim 17 wherein said tunnels are sized to receive said at least one bone engaging member.

20. The bone fixation device of claim 17, wherein the tunnels extend at an angle relative to the longitudinal axis of the rod member.

21. The bone fixation device of claim 15 wherein the one or more guide members comprise at least a portion of an external surface of the distal end portion of the rod member.

22. The bone fixation device of claim 4, wherein the main shaft of the rod member comprises receiving means to receive the at least one bone engaging member.

23. The bone fixation device of claim 22 wherein the receiving means comprises one or more channels in an outer surface of the main shaft of the rod member.

24. The bone fixation device of claim 22 wherein the receiving means comprises one or more tunnels through an interior of the main shaft of the rod member.

25. The bone fixation device of claim 1, further comprising an introducer collar.

26. The bone fixation device of claim 25 wherein the introducer collar comprises a tubular main body which is moveable both longitudinally and rotationally relative to the rod member.

27. The bone fixation device of claim 25 wherein the tubular main body of the introducer collar includes at least one receiving member to receive the at least one bone engaging member.

28. The bone fixation device of claim 25 wherein the introducer collar comprises an elongate handle.

29. The bone fixation device of claim 1 further comprising a locking member.

30. The bone fixation device of claim 29 wherein the locking member comprises a distal locking member and a proximal locking member which together secure the rod and one or more of the bone engaging members within a fractured bone when the device is in use.

31. The bone fixation device of claim 30 wherein the distal locking member comprises an elongate tubular main body extending from a first end to a second end an inner lumen sized to receive the rod member and the at least one bone engaging member.

32. The bone fixation device of claim 31 wherein an inner surface of the distal locking member comprises one or more holding members to engage the at least one bone engaging member.

33. The bone fixation device of claim 32 wherein said one or more holding members comprise two longitudinal channels on said inner surface of the distal locking member to receive two bone engaging members.

34. The bone fixation device of claim 30 wherein the distal locking member is longitudinally moveable relative to the rod member.

35. The bone fixation device of claim 31 wherein the diameter of the lumen of the distal locking member is less than the diameter of the distal end portion of the rod member.

36. The bone fixation device of claim 30 wherein the distal locking member is moveable between a non-locked configuration and a locked configuration.

37. The bone fixation device of claim 36 wherein, in the non-locked configuration, the distal locking member is slidably moveable relative to the rod member and the at least one bone engaging member and in the locked configuration, the second end of the distal locking member is in engagement with the distal end portion of the rod member.

38. The bone fixation device of claim 30 wherein the distal locking member comprises a continuous tubular main body.

39. The bone fixation device of claim 30 wherein the distal locking member comprises a plurality of inter-connectable tubular segments.

40. The bone fixation device of claim 30 wherein the proximal locking member comprises an elongate tubular main body having a lumen to receive at least a portion of the rod member.

41. The bone fixation device of claim 40 wherein the proximal locking member has an internal screw threaded portion that is engageable with a complementary screw threaded portion of the rod member.

42. The bone fixation device of claim 30 wherein the proximal locking member comprises a keyway to receive a locking pin.

43. The bone fixation device of claim 1 wherein the at least one bone engaging member is made from a resiliently flexible material.

44. The bone fixation device of claim 43 wherein the resiliently flexible material is a shape memory material.

45. A method of securing a bone fixation device in a fractured bone of a patient; the bone fixation device including a rod member that extends along a longitudinal axis from a proximal end to a distal end and at least one bone engaging member, said at least one bone engaging member extending from a first end to a second end, the method comprising:
introducing the rod member and the at least one bone engaging member into the medullary space of a fractured bone;
advancing the rod member and the at least one bone engaging member through the medullary space and across the site of fracture such that the proximal end of the rod member and the first end of the at least one bone engaging member are positioned proximal to the fracture site and the distal end of the rod member and the second end of the at least one bone engaging member are positioned distal to the fracture site; and
further advancing the at least one bone engaging member and causing at least the second end of the at least one bone engaging member to extend into a portion of cortical bone distal to the site of fracture.

46. The method of claim 45 wherein the at least one bone engaging member is advanced through the medullary space by the rod member.

47. The method of claim 45 wherein the at least one bone engaging member is advanced through the medullary space by an introducer collar.

48. The method of claim 45 wherein step further advancing the at least one bone engaging member comprises drilling.
